(19) United States
(12) Patent Application Publication
(10) Pub. No.: US 2004/0171896 A1
(43) Pub. Date: Sep. 2, 2004

(54) Preparation of compounds with the CF3 group

(75) Inventors: Max Braun, Wedemark (DE); Carsten Brosch, Hannover (DE)

Correspondence Address: CROWELL & MORING LLP, INTELLECTUAL PROPERTY GROUP, P.O. BOX 14300, WASHINGTON, DC, 20044-4300, US

(73) Assignee: Solvay Fluor und Derivate GmbH, Hannover (DE)

(21) Appl. No.: 10/790,686

(22) Filed: Mar. 3, 2004

Related U.S. Application Data
(63) Continuation of application No. PCT/EP02/09547, filed on Aug. 27, 2002.

(30) Foreign Application Priority Data
Sep. 4, 2001 (DE) 101 43 177.5

(51) Int. Cl. C07C 17/08

(52) U.S. Cl. 570/167

(57) ABSTRACT

Highly fluorinated antimony, especially in the form of a hydrogen fluoride addition compound, can be used as an isomerization catalyst for the isomerization of certain halocarbon compounds or halogenated hydrocarbon compounds. For example, 1,1,1-trifluoro-2,2-dichoroethane can be produced from 1,1,2-trifluoro-1,2-dichloroethane. The method also is suitable for the purification of certain halocarbon or halogenated hydrocarbon compounds which are contaminated by isomerizable compounds.

### CROSS REFERENCE TO RELATED APPLICATIONS

This application is a continuation of international patent application no. PCT/EP02/09547, filed Aug. 27, 2002 designating the United States of America and published in German as WO 03/020675, the entire disclosure of which is incorporated herein by reference. Priority is claimed based on Federal Republic of Germany patent application no. DE 101 43 177.5, filed Sep. 4, 2001.

### BACKGROUND OF THE INVENTION

The invention relates to a method for the preparation of compounds with a CF₃ group by making use of isomerization.

Halogenated hydrocarbon compounds can be used, for example, as refrigerants, blowing agents and also as intermediates in chemical syntheses. The compound CF₃CHCl₂, for example, is used as a component of refrigerants and blowing agents. It can also be converted by photooxidation into trifluoroacetyl chloride.

It is already known that compounds, which contain the molecular group CF₂Cl—CF, can be iomerized into compounds which contain the CF₃—CCl group. Usually, aluminum trichloride is used as an isomerization catalyst. The aim of this conversion may, for example, be the synthesis of an asymmetric compound from a symmetric compound. For example, HCFC-123 can be synthesized by isomerizing HCFC-123a over aluminum chloride. Such an isomerization may also be carried out for the purpose of purifying HCFC-123, which is contaminated, for example, by HCFC-123a.

### SUMMARY OF THE INVENTION

It is an object of the invention to provide an improved method for preparing compounds which contain a CF₃ group.

Another object of the invention is to provide a new method for the preparation of compounds which contain the CF₃ group, in which the method is carried out via an isomerization reaction.

An additional object of the invention is to provide a method for purifying compounds which contain the CF₃ group which are contaminated by isomeric contaminants.

These and other objects have been achieved in accordance with the present invention by providing a method for preparing a compound corresponding to the formula CF₃—CClXY (I) in which X represents H, Cl or F, and Y represents H, Cl, F, C1-C3 alkyl, or C1-C3 alkyl substituted by at least one halogen atom, with the proviso that X and Y cannot simultaneously represent fluorine, the method comprising contacting at least one compound selected from the group consisting of compounds corresponding to the formula CF₂Cl—CFXY (II) and compounds corresponding to the formula CFCl₂—CF₂Y (III), in which X and Y have the meanings given above, with an antimony compound corresponding to the formula SbCl_{0-0.5} F_{4.5-5}.

In accordance with the method of the invention for preparing compounds of the formula (I)

CF₃—CClXY (I)

in which X represents H, Cl or F and Y represents H, Cl, F, C1-C3 alkyl, or C1-C3 alkyl substituted by at least one halogen atom, with the proviso that X and Y cannot simultaneously represent fluorine,

compounds of the formula (II)

CF₂Cl—CFXY (II)

or compounds of the formula (III)

CFCl₂—CF₂Y (III),

in which X and Y have the meanings given above, are contacted with the antimony compound SbCl_{0-0.5}F_{4.5-5}. Advantageously, HF is also present at the same time. If, in this case, the sum of the HF and the antimony compound in the reaction mixture is 100 parts by weight, the HF is contained in an amount of 1 to 70 parts by weight and preferably of 20 to 50 parts by weight, the remainder up to 100 parts by weight being the antimony compound. If, in accordance with a preferred embodiment, HF is present, it may be assumed that the antimony compounds will be present in the form of HF adducts. In this case, contacting is carried out in the presence of HF adducts of SbCl_{0-0.5}F_{4-4.5}. A portion of not more than 10 mole percent of the antimony compound can also be replaced by Sb(III), for example, by SbF₃.

Preferably, the contacting is carried out with antimony pentafluoride (in this connection, it should be kept in mind that, in the presence of HF, adducts of the antimony(V) compound are present, as mentioned above). Antimony pentafluoride is a commercially available product. The highly fluorinated SbCl_{0-0.5}F_{4.5} can also be prepared from SbCl₅ or SbCl₃ and chlorine, as well as sufficient HF and SbF₅ as described in Schulz et al., U.S. Pat. No. 6,645,451 (=EP 816,287).

The isomerization is carried out at a temperature ranging from 20° to 150° C., and preferably from 50° to 120° C. The pressure depends on the composition of the mixture and advantageously ranges from atmospheric pressure to 6 bar. The temperature and pressure are selected, in particular, so that the contacting is carried out in the liquid phase.

The molar ratio of the compound of formula (II), the compound of formula (III), or the sum of the two compounds when both are present, to the antimony compound preferably ranges from 0.1:1 to 10:1, and particularly preferably from 0.5:1 to 2:1.

Advantageously, the isomerization is carried out in a corrosion-resistant apparatus. Reactors and equipment parts which consist of aluminum or polytetrafluoroethylene (Teflon™) or are coated with aluminum or polytetrafluoroethylene (Teflon™) have proven to be particularly corrosion-resistant. In the case reactors made of metallic materials, a pre-fluorination with HF or F₂ carried out before the start of the reaction has been shown to have a positive effect on the corrosion behavior. Equipment made from alloys such as those described in Braun et al., WO 03/054241 (=DE 101 63 171) is very suitable. These alloys contain at least 3.5% by weight of aluminum, as well as nickel and/or silicon. Alloys are preferred which consist of or contain 80 to 92% by weight of aluminum and 8 to 20% by weight of silicon or which contain or consist of 3.5 to 10% by weight of aluminum and 90 to 96.5% by weight of nickel.

The method of the invention can be carried out batchwise or continuously. The reaction can be conducted continuously in a bubble reactor, whereby the educt, or if HF is used at the same time, a mixture of HF and educt, is passed into the liquid antimony compound, and a mixture of product and HF is discharged. In the case of HCFC-123, the product mixture is an azeotrope with HF, which, upon cooling, separates in the liquid phase into two phases.

It is well known that Sb(V) halide decomposes with formation of Sb(III) halide and halogen. If desired, Sb (II) halide can be converted once again into Sb(V) halide with chlorine or fluorine. This can be done batchwise or continuously, for example, with constant removal of a portion of the reaction mixture and treatment of the latter with halogen.

The invention thus provides a novel method of effecting isomerization. The palette of usable isomerization catalysts is accordingly expanded.

### EXAMPLE

The following example is intended to illustrate and explain the invention in further detail without limiting its scope.

### Example I

Synthesis of Isomerically Pure 1,1,1-trifluoro-2,2-dichloroethane (123) by Catalytic Isomerization of 1,1,2-trifluoro-1,2-dichloroethane (123a) and 1,2,2-trifluoro-1,1-dicholoroethane (123b) Contained Therein, with Fluorinated Sb Catalysts.

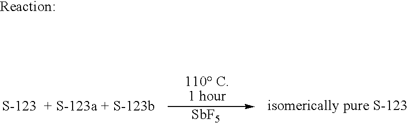

Formulation:

| | Molecular | | |
|---|---|---|---|
| Material | Weight | Weight in g | Moles |
| 123 | 153.00 | 38.25 | 0.25 |
| SbF₅ | 216.75 | 11.88 | 0.05 |
| HF | 20.00 | 9.37 | 0.47 |

Procedure:

A mixture of 89.9% of 123, 10.0% of 123a and 0.1% of 123b was added to an autoclave with a Teflon™ liner and mixed in a ratio of 1:2 with the catalyst mixture. The composition of the catalyst was as follows:

55.47% by weight of SbF₅

0.64% by weight of SbF₃

43.82% HF

0.07% by weight of HCl

The autoclave was closed, heated to 110° C. and maintained at this temperature for one hour at a pressure of about five bar. It is assumed that an HF adduct of antimony pantafluoride was present in the reactor. Subsequently the autoclave was cooled with ice, and the pressure in the gas phase was relieved. The contents of the autoclave were hydrolyzed in ice/tartaric acid (tartaric acid because of the solubility of the Sb complex) (closed system) and the organic phase was separated from the aqueous phase. The purity of the 123 isomer isolated was 99.9%; in addition, 0.14% of newly formed 1,1,2-trifluoro-1,2,2-trichloroethane (113) were present. An additional Sb′″ content of 1% was detected by the analysis of the aqueous phase treated with tartaric acid.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A method for preparing a compound corresponding to formula (I)
CF₃—CClXY (I)
wherein
X represents H, Cl or F, and
Y represents H, Cl, F, C1-C3 alkyl, or C1-C3 alkyl substituted by at least one halogen atom,
with the proviso that X and Y cannot simultaneously represent fluorine,
said method comprising contacting at least one compound selected from the group consisting of compounds corresponding to formula (II)
CF₂Cl—CFXY (II)
and compounds corresponding to formula (III)
CFCl₂—CF₂Y (III),
in which X and Y have the meanings given above,
with an antimony compound corresponding to the formula SbCl_{0-0.5} F_{4.5-5}.

2. A method according to claim 1, wherein X and Y each represent Cl.

3. A method according to claim 1, wherein X represents H, and Y represents Cl.

4. A method according to claim 1, wherein said method is carried out at a temperature ranging from 20° to 150° C.

5. A method according to claim 4, wherein said method is carried out at a temperature ranging from 50° to 120° C.

6. A method according to claim 1, wherein the molar ratio of the compound of formula (II) or the compound of formula (III) or of the sum of the compounds of formula (II) and formula (III), to the antimony compound lies in the range from 0.1:1 to 10:1.

7. A method according to claim 6, wherein the molar ratio lies in the range from 0.5:1 to 2:1.

8. A method of purifying a compound corresponding to formula (I)
CF₃—CClXY (I)
contaminated with at least one compound selected from the group consisting of compounds of formula (II)
CF₂Cl—CFXY (II)
and compounds of formula (III)
CFCl₂—CF₂Y (III),
said method comprising treating the contaminated compound of formula (I) according to the method of claim 1.

9. A method according to claim 8, wherein the contaminated compound of formula (I) comprises 1,1,1-trifluoro-2,2-dichloroethane contaminated with one or both of 1,1,2-trifluoro-1,2-dichloroethane and 1,2,2-trifluoro-1,1-dichloroethane.

10. A method according to claim 1, wherein said method is carried out in a corrosion-resistant apparatus.

11. A method according to claim 10, wherein the apparatus is coated with or made of aluminum.

12. A method according to claim 10, wherein the apparatus is coated with or made of polytetrafluoroethylene.

13. A method according to claim 1, wherein the contacting is carried out in liquid phase.

14. A method according to claim 1, wherein said method is carried out batchwise.

15. A method according to claim 1, wherein said method is carried out continuously.

16. A method according to claim 1, wherein said method is carried out in a reaction mixture containing 1 to 70 parts by weight of HF and 30 by 99 parts by weight of antimony compound, whereby the sum of the HF and the antimony compound amounts to 100 parts by weight.

17. A method according to claim 1, wherein said method is carried out in a reactor having an inner surface which is at least partially fluorinated.

18. A method according to claim 1, wherein said method is carried out in the presence of HF and the antimony compound is present in form of an HF adduct.
